SEQUENCE LISTING (18 sequences)

SEQ ID NO: 1 - DNA, 17 bp, Artificial
  synthesized
atcaagaaggtggtgaa

SEQ ID NO: 2 - DNA, 33 bp, Artificial
  probe
tgctcagtgtagcccattcaccaccttcttgat

SEQ ID NO: 3 - DNA, 16 bp, Artificial
  probe
tgggctacactgagca

SEQ ID NO: 4 - DNA, 36 bp, Artificial
  probe
tgactgcgagtagtagccattcaccaccttcttgat

SEQ ID NO: 5 - DNA, 19 bp, Artificial
  probe
tggctactactcgcagtca

SEQ ID NO: 6 - DNA, 29 bp, Artificial
  probe
tctcaggtaccattcaccaccttcttgat

SEQ ID NO: 7 - DNA, 12 bp, Artificial
  probe
tggtacctgaga

SEQ ID NO: 8 - DNA, 17 bp, Artificial
  probe
ccaccgtttttcctcag

SEQ ID NO: 9 - DNA, 33 bp, Artificial
  probe
tgctcagtgtagcccactgaggaaaaacggtgg

SEQ ID NO: 10 - DNA, 36 bp, Artificial
  probe
tgactgcgagtagtagccactgaggaaaaacggtgg

SEQ ID NO: 11 - DNA, 29 bp, Artificial
  probe
tctcaggtaccactgaggaaaaacggtgg

SEQ ID NO: 12 - DNA, 17 bp, Artificial
  probe
atcttaaatcgcaaggt

SEQ ID NO: 13 - DNA, 33 bp, Artificial
  probe
tgctcagtgtagcccaaccttgcgatttaagat

SEQ ID NO: 14 - DNA, 36 bp, Artificial
  probe
tgactgcgagtagtagccaaccttgcgatttaagat

SEQ ID NO: 15 - DNA, 29 bp, Artificial
  probe
tctcaggtaccaaccttgcgatttaagat

SEQ ID NO: 16 - DNA, 17 bp, Artificial
  probe
atcccgaaggtggttac

SEQ ID NO: 17 - DNA, 29 bp, Artificial
  probe
ggtaccatctcagtaaccaccttcgggat

SEQ ID NO: 18 - DNA, 12 bp, Artificial
  probe
tgagatggtacc

(19) United States
(12) Patent Application Publication
(10) Pub. No.: US 2021/0214777 A1
(43) Pub. Date: Jul. 15, 2021

(54) KIT AND METHOD FOR SIMULTANEOUSLY DETECTING DROPLET DRIFT OR DEPOSITION OF MULTIPLE SPRAYS

(71) Applicant: CHINA AGRICULTURAL UNIVERSITY, Beijing (CN)

(72) Inventors: Jianli SONG, Beijing (CN); Yang LIU, Beijing (CN); Xiongkui HE, Beijing (CN); Zhenhua ZHANG, Beijing (CN); Sen PANG, Beijing (CN); Shaoqing XU, Beijing (CN); Guangyu WANG, Beijing (CN); Zongyang LI, Beijing (CN); Xuemin WU, Beijing (CN); Xuefeng LI, Beijing (CN)

(21) Appl. No.: 17/214,743

(22) Filed: Mar. 26, 2021

Related U.S. Application Data
(63) Continuation-in-part of application No. PCT/CN2019/108036, filed on Sep. 26, 2019.

(30) Foreign Application Priority Data
Sep. 26, 2018 (CN) 201811120935.8

(51) Int. Cl. C12Q 1/6834; G01N 21/64

(52) U.S. Cl. CPC C12Q 1/6834; G01N 2021/6439; G01N 21/6428

(57) ABSTRACT

A kit for simultaneously detecting the droplet drift or deposition of multiple sprays includes detection membranes fixed with immobilized probes, transition probes capable of specifically binding to the immobilized probes, and biotinylated chromogenic probes capable of specifically binding to the transition probes. The transition probes are added to the spray liquids as tracers. After spraying, the transition probes specifically bind to the immobilized probes on the detection membranes. The biotinylated chromogenic probes bind to the transition probes through hybridization. After the chromogenic treatment, the droplet volume is determined according to the color depth, and the spray deposition parameters of droplets are determined according to the location and size of colored spots.

### CROSS-REFERENCE TO RELATED APPLICATIONS

The present application is a continuation in part application of PCT international application no. PCT/CN2019/108036, filed on Sep. 26, 2019, which claims the priority of Chinese Patent Application No. 201811120935.8 filed on Sep. 26, 2018 with China National Intellectual Property Administration and entitled “KIT AND METHOD FOR SIMULTANEOUSLY DETECTING DROPLET DRIFT OR DEPOSITION OF MULTIPLE SPRAYS”, which is incorporated herein by reference in its entirety.

### INCORPORATION OF SEQUENCE LISTING

This application contains a sequence listing submitted in Computer Readable Form (CRF). The CFR file containing the sequence listing entitled “PA150-0108_ST25.txt”, which was created on Mar. 26, 2021, and is 3,389 bytes in size. The information in the sequence listing is incorporated herein by reference in its entirety.

### TECHNICAL FIELD

The present invention relates to the technical field of spray droplet detection in agricultural, in particular to a reverse dot blot kit and detection method developed for simultaneous, qualitative and quantitative detection of droplet deposition of multiple sprays such as pesticides, fertilizers, and water.

### BACKGROUND

It is always a problem during the agricultural development process that how to produce more crops of good quality with minimized area of arable land, especially in present days where arable land is gradually reducing. According to statistics, in only one year from 2015 to 2016, arable land in China decreased by 1.153 million Mu [ [symbol] ]. To ensure a stable or increased yield of agricultural products, proper irrigation, fertilization and pesticide application are the most important procedures in modern agriculture. “Integrated plantation and protection” is one of the latest plantation schemes, which means a series of proper, programmed and controllable procedures from seed selection and germination, through irrigation, fertilization and pesticide application and to final picking, in the whole plantation and cultivation process, so as to optimize the agricultural production. How to simultaneously control complex procedures such as irrigation, fertilization and pesticide application in a simple and efficient manner provides guidelines for plantation. The three procedures are mostly implemented by applying sprays with agricultural plant protection machinery, which raises high requirements on the method for monitoring and detecting the size, distribution and the drift/deposition volume of the droplets. Common methods for detecting droplets include direct detection method, tracer method, and direct observation method.

In the direct detection method, samples are collected after spraying, subjected to extraction, purification and concentration, the active ingredient on the samples is determined through HPLC-MS, GC-MS and other devices, and then the deposition volume is calculated. Despite having high precision and being capable of detecting multiple active substances in one detection, the method cannot be applied to the detection of some inorganic fertilizers, and cannot provide more information other than the deposition volume, such as the droplet size and distribution.

In the tracer method, a tracer is added to the spray liquid, samples are collected after spraying, the amount of the tracer on the target is determined through instrumental analysis, and then the pesticide deposition volume on the target is calculated. Common tracers include water-soluble dyes such as tartrazine and allura red and fluorescent tracers such as brilliant sulphoflavine (B S F) and pyranin. The tracer method has the advantages of rapid detection, cost-efficiency and low requirement on reagent storage, and is a common method for detecting pesticide deposition. However, the precision of the method is susceptible to the properties of tracers and thus is not good, and may cause color contamination for the crops and the operators as tracers are colored agents. In the meantime, as there is no specific detection method for each tracer, the above tracers cannot be distinguished well in simultaneously thus cannot achieve the detection of multiple different spray liquids or suspensions.

In the direct observation method, droplets are collected with water-sensitive paper, oil-sensitive paper, Kromekote® cards and other droplet collectors, and then are subjected to image processing to observe parameters such as droplet size and distribution, or the droplets are subjected to direct observation for droplet properties in direct observation instruments such as a laser particle size analyzer. Using water-sensitive paper is one of the commonly used methods for detecting spray droplets in the industry. The method can detect the droplet deposition distribution on site, but the water-sensitive paper changes color when contacting with moisture and is thus susceptible to the environment. Thus, the method is inapplicable in rainy days or in a condition of high humidity. The method is also incapable of quantitative analysis of droplets. Furthermore, the method detects the sprayed dispersion (water or oil) and is not specific, and thus cannot achieve the simultaneous detection of multiple samples.

None of the three methods in the prior art is capable of providing information such as the droplet size and distribution, accurately quantifying the drift/deposition volume and simultaneously detecting multiple samples at the same time.

Reverse dot blot (RDB) is a common DNA detection technique, and utilizes the specific binding of DNA sequence, immobilizes the complementary strand of a target DNA to be detected on a substrate; the complementary strand binds to a DNA sample to be detected after extraction and amplification, and captures a biotinylated DNA to be detected after amplification, thus achieving the detection of the sample. RDB is mainly used in the prior art for detecting natural short chains of nucleic acid, but is not used in detecting artificial characteristic sequence of single-stranded deoxyribonucleic acid. For the detection of agricultural spraying, due to the complicated field environment, natural short chains of nucleic acid may interfere with the detection, leading to false positive or false negative results and affecting the quantitative analysis. Thus natural short chains are not applicable for detecting agricultural spraying.

### SUMMARY

The objective of the present invention is to provide a kit for simultaneously detecting the droplet deposition of multiple agricultural sprays by using reverse dot blot technology, thereby realizing the rapid, accurate, easy-to-operate, cost-efficient and simultaneous detection of droplet distribution of multiple liquid sprays.

The present invention firstly provides a method for simultaneously detecting the droplet deposition volumes of multiple sprays. As shown in FIG. 1, the process comprises 5 procedures: preparation of detection membrane, preparation of spray liquid, spraying, establishment of standard curve, and chromogenic treatment. The specific process is as follows: based on the binding specificity of single-stranded deoxyribonucleic acids with different characteristic sequences, a series of single-stranded deoxyribonucleic acids with different characteristic sequences are designed as immobilized probes and then fixed on a substrate to prepare a variety of detection membrane. Subsequently, the corresponding single-stranded deoxyribonucleic acids with different characteristic sequences are added into different spray liquids as tracers. After spraying, the detection membrane is retrieved, and the droplet information such as droplet size and distribution of the different spray liquids is obtained after signal amplification and chromogenic treatment. Finally, the droplet deposition is calculated by computer image processing software.

Specifically, the method for simultaneously detecting the droplet drift or deposition volumes of multiple sprays disclosed herein comprises the following steps:

(1) adding different transition probes into multiple spray liquids respectively as tracers, with only one transition probe being added to each spray liquid;

(2) applying the spray liquids containing transition probes, then the transition probes in the spray liquids specifically binding to the corresponding immobilized probes on the detection membranes, wherein the detection membranes are substrates carrying the immobilized probes;

(3) adding biotinylated chromogenic probes, then they binding to the corresponding transition probes through hybridization, after the chromogenic treatment, determining the volume of droplets according to the color depth, and determining the spray drift or deposition according to the location and size of colored spots.

The transition probes are not biotinylated, but have nucleotide sequences capable of complementarily pairing with the corresponding immobilized probes and the corresponding chromogenic probes. The immobilized probes do not specifically bind to the chromogenic probes, and different transition probes do not specifically bind to each other.

The transition probes and the immobilized probes are single-stranded deoxyribonucleic acids with characteristic sequences, wherein the length of the transition probes is 24-50 nt, and the length of the immobilized probes is 12-25 nt. One end of the immobilized probes is amino-modified and covalently binds to an exposed carboxyl of the substrate.

In the method disclosed herein, the complementary pairing region of the chromogenic probe and the transition probe is of 15-40 nt. If the immobilized probe is 5′-labeled, the chromogenic probe is 3′-biotinylated; and if the immobilized probe is 3′-labeled, the chromogenic probe is 5′-biotinylated.

In the method disclosed herein, the length of the immobilized probe is preferably 18-20 nt, and the complementary pairing region of the transition probe and the immobilized probe is preferably of 15-25 nt.

In step (2), the major reagents used include: 0.1-0.3 M (preferably 0.1 M) HCl solution, 10-20% (preferably 15%) EDC (1-(3-dimethylaminopropyl)-3-ethylcarbodiimide) solution, 0.025-0.2 μM (preferably 0.03 μM) immobilized probe solution, 0.3-1.0 M (preferably 0.5 M) NaHCO₃ solution, and 0.05-0.5 M (preferably 0.2 M) NaOH solution.

The detection membrane in step (2) is prepared according to the following method: acquiring a substrate of a required area, treating the substrate with 0.1-0.3 M HCl, and washing; incubating the substrate in 10-20% EDC solution and washing; incubating the substrate in 0.3-1.0 M NaHCO₃ solution containing 0.025-0.2 μM immobilized probe; and incubating the treated substrate in NaOH solution, washing and drying.

Preferably, the detection membrane is prepared according to the following method: acquiring a substrate of a required area, treating the substrate with 0.1 M HCl, and washing; incubating the substrate in 15% EDC for 0.5-1 h and washing; incubating the substrate in 0.5 M NaHCO₃ solution containing 0.03 μM immobilized probe for 10-20 min; and incubating the treated substrate in 0.05-0.5 M NaOH solution for 5-15 min, washing and drying. The substrate is a nitrocellulose membrane, a nylon membrane, a carboxylated organic glass film or a carboxylated polypropylene plastic film.

The spray liquid in step (2) may be a pesticide formulation, a liquid fertilizer, other liquid formulations or water. The spray liquid has the following formula: during the prepareation of the spray liquid, it mainly comprises: 0-60% of pesticide or liquid fertilizer (or water), 0.025-0.1 μM (preferably 0.060 μM) transition probe, 0-0.045 mol/L ion buffer, and 0-0.15% of surfactant (if pesticide or fertilizer is used, the transition probe can be directly added because the pesticide or fertilizer itself contains surfactant and ion buffer; if water is used as the spray liquid, a certain amount of ion buffer and surfactant should be added). Major formulations include water-based formulation and oil-based formulation.

The pesticide formulation includes water-based formulation, oil-based formulation, wettable powder, microcapsule, water-based suspension, oil-based suspension and the like. The pesticide includes insecticides, fungicides, herbicides, acaricides, nematicides and the like.

The liquid fertilizer includes solution, suspension, foliar fertilizer and the like, wherein the fertilizer includes any one of a nitrogenous fertilizer, a phosphate fertilizer or a potassic fertilizer, or a compound fertilizer comprising two or more thereof.

The transition probe is a single-stranded deoxyribonucleic acid with a characteristic sequence of 24-50 nt (preferably 36-40 nt).

The ion buffer is a buffer prepared from one or more inorganic salts and/or organic salts, wherein the anion of the solution is one or more of carbonate, bicarbonate, phosphate, hydrogen phosphate, dihydrogen phosphate, citrate, dihydrogen citrate and the like, and the cation is one or more of potassium ion, sodium ion, lithium ion, calcium ion and the like.

The surfactant is one or more of sodium alkyl sulfonate, nekal, tea seed powder, Gleditsia sinensis extract powder, SDS (sodium dodecyl sulfate), Morwet EFW (sodium butylnaphthalene sulfonate), TERWET 1004 and the like.

In step (3) of the method disclosed herein, the reagents used include: hybridization buffer, washing buffer, 0.05-0.20 μM chromogenic probe solution, catalase solution and TMB single-component solution.

The hybridization buffer mainly contains 0.02-0.045 mol/L ion buffer and 0.06-0.15% of surfactant.

The washing buffer mainly contains 5.0-10.0 mol/L ion buffer and 0.02-0.20% of surfactant.

The ion buffer is a buffer prepared from one or more inorganic salts and/or organic salts, wherein the anion of the solution is one or more of carbonate, bicarbonate, phosphate, hydrogen phosphate, dihydrogen phosphate, citrate, dihydrogen citrate and the like, and the cation is one or more of potassium ion, sodium ion, lithium ion, calcium ion and the like.

The surfactant is one or more of sodium alkyl sulfonate, nekal, tea seed powder, Gleditsia sinensis extract powder, SDS (sodium dodecyl sulfate), Morwet EFW (sodium butylnaphthalene sulfonate), TERWET 1004 and the like.

The chromogenic probe is a single-stranded deoxyribonucleic acid with a characteristic sequence of 12-25 nt (preferably 18-20 nt).

The TMB single-component solution mainly comprises: 0.5-2.0 mM (preferably 1.0 mM) TMB (3,3′,5,5′-tetramethylbenzidine), 0.5-2.0 mM (preferably 1.0 mM) oxidant, 150-300 mM (preferably 200 mM) ion buffer, and 0.1-0.5 mM stabilizer. The preparation process is as follows: solution a: weighing TMB and stabilizer and adding DMSO to dissolve them; solution b: preparing an ion buffer with deionized water, adding oxidant, and adjusting the pH to 4.0-6.0 with hydrochloric acid; and mixing solution a and solution b in a certain ratio to give the TMB single-component solution before use.

The oxidant is one or more of hydrogen peroxide, urea-hydrogen peroxide, peroxyacetic acid, tert-butyl hydroperoxide, dimethyl dioxirane and the like.

The ion buffer is a buffer prepared from one or more inorganic salts and/or organic salts, wherein the anion of the solution is one or more of carbonate, bicarbonate, phosphate, hydrogen phosphate, dihydrogen phosphate, citrate, dihydrogen citrate and the like, and the cation is one or more of potassium ion, sodium ion, lithium ion, calcium ion and the like.

The stabilizer is one or more of sodium borohydride, sodium cyanoborohydride, tetrabutylammonium borohydride (TBABH), lithium tri-sec-butylborohydride, lithium borohydride and the like.

In some embodiments of the present invention, the method for simultaneously detecting the droplet drift or deposition volumes of multiple sprays is as follows:

1) Preparation of the detection membrane: acquiring a carboxylated nylon membrane of a required area, treating the membrane with 0.1 M HCl, and washing; incubating the membrane in 15% EDC for 1 h and washing; incubating the membrane in 0.5 M NaHCO₃ solution containing 0.03 μM immobilized probe for 20 min; and incubating the treated membrane in 0.2 M NaOH solution for 5-15 min, washing and drying. Different detection membrane may be prepared in this step using different immobilized probes.

2) Spraying: preparing a spray liquid by adding a pesticide formulation, a liquid fertilizer or water into a dosing tank, adding 0.025-0.1 μM transition probes, and based on the requirements of pesticide formulation or sprayer, adding 0-0.15% of surfactant and 0-0.045 mol/L ion buffer to prepare a transition probe spray liquid. The transition probes added are selected according to the immobilized probes. Detection membranes containing different immobilized probes are arranged on the target crops, and are retrieved after spraying for chromogenic treatment.

3) Chromogenic treatment: incubating the detection membranes sprayed with the spray liquids containing transition probes in a hybridization buffer at 30-40° C. for 25-40 min, and washing the detection membranes in 50 mL of hybridization buffer for 2 min; transferring the detection membrane into the hybridization buffers containing corresponding chromogenic probes for chromogenic reaction for 5-15 min at 30-40° C.; transferring the detection membrane into 50 mL of washing buffer and washing 3 times; transferring the detection membrane into 50 mL of hybridization buffer for a 2-min washing; adding 15 μL of streptavidin-labeled horseradish peroxidase into a hybridization buffer to prepare an enzyme solution, and incubating the detection membrane in the enzyme solution for enzyme-linked reaction at 37° C. for 15-20 min; washing the detection membranes with hybridization buffer, transferring the detection membrane into the TMB single-component solution for chromogenic reaction, wherein the TMB single-component solution is catalyzed by the horseradish peroxidase bound to the detection membrane, and then color develops on the detection membrane; and after 3 min, washing the membrane with water to terminate the reaction, and drying the membrane. Information such as the distribution and size of droplets can be directly observed through the chromogenic reaction. Finally, an image file is obtained by photographing or scanning. Gray values of unit areas are obtained through an image processing software (for example, Photoshop, Image J and the like), and a total gray value of a selected area is calculated. The deposition volume is calculated from a standard curve.

4) Establishment of standard curve: selecting 5 detection membranes containing immobilized probes, applying 0.5 μL of spray liquid containing transition probe on 1, 2, 3, 4 and 5 spots on the 5 detection membranes respectively, wherein the volumes of the spray liquid containing transition probe on the 5 detection membranes are 0.5 μL, 1.0 μL, 1.5 μL, 2.0 μL and 2.5 μL, respectively. Another detection membrane is taken as the blank background. An image file is obtained by photographing or scanning. Gray values of unit areas are obtained through an image processing software (for example, Photoshop, Image J and the like), and a total gray value of a selected area is calculated. Finally, a standard curve of the total gray value as the ordinate against the volume of transition probe solution as the abscissa is plotted, and a corresponding linear equation is calculated.

Based on the detection method disclosed herein, the present invention further provides a kit for simultaneously detecting the droplet drift or deposition volumes of multiple sprays, comprising detection membranes, transition probes and chromogenic probes, wherein the numbers of the detection membranes, the transition probes and the chromogenic probes are all >2 and are different;

The detection membrane is a substrate fixed with the immobilized probe. The length of the immobilized probe is 12-25 nt. One end of the immobilized probe is amino-modified and covalently binds to an exposed carboxyl group of the substrate. The substrate is a material with exposed carboxyl groups;

The length of the transition probe is 24-50 nt. The 3′ or 5′ end of the chromogenic probe is biotinylated. The chromogenic probe can specifically bind to the transition probe but cannot specifically bind to the immobilized probe.

The length of the chromogenic probe is 12-25 nt.

The catalase is a solution of streptavidin-labeled horseradish peroxidase.

The TMB single-component solution mainly comprises: 0.5-2.0 mM (preferably 1.0 mM) TMB (3,3′,5,5′-tetramethylbenzidine), 0.5-2.0 mM (preferably 1.0 mM) oxidant, 150-300 mM (preferably 200 mM) ion buffer, and 0.1-0.5 mM stabilizer. The preparation process is as follows: solution a: weighing TMB and stabilizer, and adding DMSO to dissolve them; solution b: preparing an ion buffer with deionized water, adding oxidant, and adjusting the pH to 4.0-6.0 with hydrochloric acid; and mixing solution a and solution b in a certain ratio to give the TMB single-component solution before use.

The oxidant is one or more of hydrogen peroxide, urea-hydrogen peroxide, peroxyacetic acid, tert-butyl hydroperoxide, dimethyl dioxirane and the like.

The ion buffer is a buffer prepared from one or more inorganic salts and/or organic salts, wherein the anion of the solution is one or more of carbonate, bicarbonate, phosphate, hydrogen phosphate, dihydrogen phosphate, citrate, dihydrogen citrate and the like, and the cation is one or more of potassium ion, sodium ion, lithium ion, calcium ion and the like.

The stabilizer is one or more of sodium borohydride, sodium cyanoborohydride, tetrabutylammonium borohydride (TBABH), lithium tri-sec-butylborohydride, lithium borohydride and the like.

The method of the present invention overcomes the limitation that the existing spray droplet detection methods cannot simultaneously detect multiple spray liquids. By utilizing the binding specificity of single-stranded deoxyribonucleic acids with different characteristic sequences, when spray droplets containing different transition probes are applied to the detection membranes, the transition probes can only bind to corresponding complementary immobilized probes fixed in a certain detection membrane, but not to the other detection membranes. The different detection membranes are subjected to chromogenic detection by using different chromogenic probes, and then various information of different droplets can be obtained by computer software. The detection method disclosed herein has the following beneficial effects: (1) it solves the problem of lacking specificity and selectivity of existing tracers; (2) it can deals with complicated situations such as simultaneous detection after sequential applications of multiple pesticide sprays or combined applications of pesticides and fertilizers; (3) it solves the problem of color contamination of water-soluble dyes and fluorescent tracers to environment by introducing transition probes as tracers, which are colorless and tasteless and pose no pollution to the environment; and (4) it simultaneously provides qualitative and quantitative information of droplet deposition through only one detection.

In summary, the present invention can solve the problems of detecting complicated situations when applying water, fertilizer and pesticide sprays in the modern agricultural production process. Based on the specific binding of single-stranded deoxyribonucleic acids with different characteristic sequences, characteristic information of all spray droplets can be obtained through one detection, and quantitative detection of droplet deposition volume can be realized after computer software processing.

### BRIEF DESCRIPTION OF THE DRAWINGS

FIG. 1 is a schematic flow chart of the three-stage reverse dot blot for detecting droplet deposition distribution of multiple sprays.

FIG. 2A illustrates the images of 5 detection membranes containing immobilized probes, and FIG. 2B illustrates the result of standard curve establishment according to the detection method disclosed herein.

FIG. 3 illustrates the experiment for simultaneously detecting the droplet properties by two probes in Example 2.

FIG. 4 illustrates the experiment for simultaneously detecting the droplet properties by three probes in Example 3.

FIG. 5 illustrates the experiment for simultaneously detecting the droplet properties by four probes in Example 4.

### DETAILED DESCRIPTION

The present invention will be further illustrated below with reference to examples, which should not be construed as limiting the present invention. Modifications or substitutions to the methods, procedures or conditions of the present invention may be implemented without departing from the spirit and scope of the present invention.

Unless otherwise specified, the techniques used in the examples are conventional techniques well known to those skilled in the art.

### Example 1. Method for Simultaneously Detecting the Droplet Drift or Deposition Volumes of Multiple Sprays

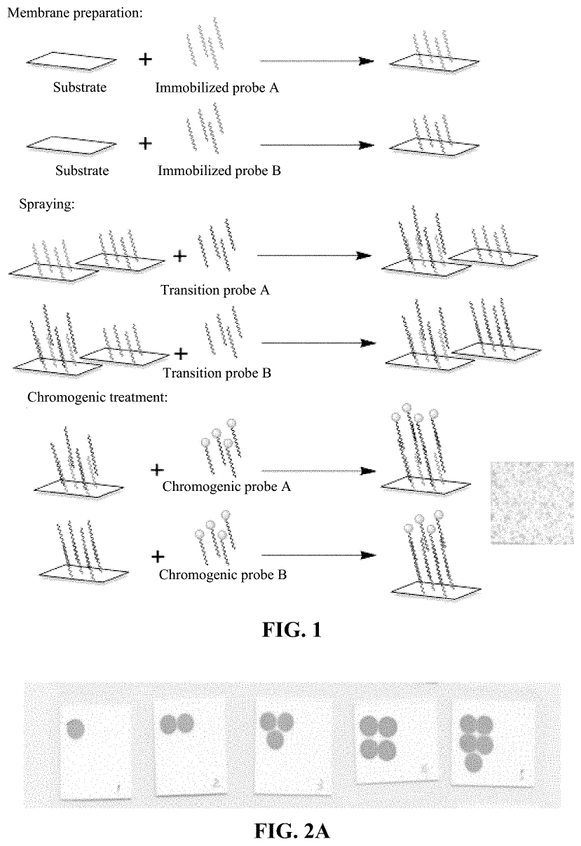

As shown in FIG. 1, the process comprises 5 procedures: preparation of detection membrane, preparation of spray liquid, spraying, establishment of standard curve, and chromogenic treatment. The specific process is as follows: based on the binding specificity of single-stranded deoxyribonucleic acids with different characteristic sequences, a series of single-stranded deoxyribonucleic acids with different characteristic sequences were designed as mobilized probes and then fixed on a substrate (a nylon membrane in this example) to prepare a variety of target membranes. Subsequently, the corresponding single-stranded deoxyribonucleic acids with different characteristic sequences were added into different spray liquids as tracers. After spraying, the substrate was retrieved, and information such as droplet size and distribution of the different spray liquids was obtained after signal amplification and chromogenic treatment. Finally, the deposition volume was calculated by computer image processing software.

1. Determination of Probes

The length of the immobilized probes was 12-25 nt, preferably 18-20 nt. One end of the immobilized probes was amino-modified, and the other end covalently bound to an exposed carboxyl of the substrate.

The transition probes were single-stranded deoxyribonucleic acids with characteristic sequences of 24-50 nt (preferably 36-40 nt) without biotinylation. The complementary pairing region of the transition probes and the immobilized probes was of 15-25 nt.

The chromogenic probes were single-stranded deoxyribonucleic acids with characteristic sequences of 12-25 nt (preferably 18-20 nt). The complementary pairing region of the chromogenic probes and the transition probes was of 15-40 nt. If the immobilized probes were 5′-labeled, the chromogenic probes were 3′-biotinylated; and if the immobilized probes were 3′-labeled, the chromogenic probes were 5′-biotinylated.

The chromogenic probes could specifically bind to the transition probes but could not specifically bind to the immobilized probes. The sequences of three probes in Table 1 are exemplary probe sequences used in the example. In addition to the nucleotide sequences of the probes in Table 1, any single-stranded deoxyribonucleic acid sequence that satisfies the above requirements can be used as the probes in the present invention.

TABLE 1

| | | |
|---|---|---|
| 1 | Immobi- | 5′-NH₂-ATCAAGAAGGTGGTGAA-3′ |
| | lized | |
| | probe 1 | |
| | Transi- | 5′-TGCTCAGTGTAGCCCATTCACCACCTTCTTGAT- |
| | tion | 3′ |
| | probe 1 | |
| | Chromo- | 5′ TGGGCTACACTGAGCA-Biotin-3′ |
| | genic | |
| | probe 1 | |
| 2 | Immobi- | 5′-NH₂-ATCAAGAAGGTGGTGAA-3′ |
| | lized | |
| | probe 1 | |
| | Transi- | 5′- |
| | tion | TGACTGCGAGTAGTAGCCATTCACCACCTTCTTGAT- |
| | probe | 3′ |
| | 1-2 | |
| | Chromo- | 5′ TGGCTACTACTCGCAGTCA-Biotin-3′ |
| | genic | |
| | probe 2 | |
| 3 | Immobi- | 5′-NH₂-ATCAAGAAGGTGGTGAA-3′ |
| | lized | |
| | probe 1 | |
| | Transi- | 5′-TCTCAGGTACCA TTCACCACCTTCTTGAT-3′ |
| | tion | |
| | probe | |
| | 1-3 | |
| | Chromo- | 5′ TGGTACCTGAGA-Biotin-3′ |
| | genic | |
| | probe 3 | |
| 4 | Immobi- | 5′-NH₂-CCACCGTTTTTCCTCAG-3′ |
| | lized | |
| | probe 2 | |
| | Transi- | 5′-TGCTCAGTGTAGCCCACTGAGGAAAAACGGTGG- |
| | tion | 3′ |
| | probe 2 | |
| | Chromo- | 5′ TGGGCTACACTGAGCA-Biotin-3′ |
| | genic | |
| | probe 1 | |
| 5 | Immobi- | 5′-NH₂-CCACCGTTTTTCCTCAG-3′ |
| | lized | |
| | probe 2 | |
| | Transi- | 5′- |
| | tion | TGACTGCGAGTAGTAGCCACTGAGGAAAAACGGTGG- |
| | probe | 3′ |
| | 2-2 | |
| | Chromo- | 5′ TGGCTACTACTCGCAGTCA-Biotin-3′ |
| | genic | |
| | probe 2 | |
| 6 | Immobi- | 5′-NH₂-CCACCGTTTTTCCTCAG-3′ |
| | lized | |
| | probe 2 | |
| | Transi- | 5′-TCTCAGGTACCA CTGAGGAAAAACGGTGG-3′ |
| | tion | |
| | probe | |
| | 2-3 | |
| | Chromo- | 5′ TGGTACCTGAGA-Biotin-3′ |
| | genic | |
| | probe 3 | |
| 7 | Immobi- | 5′-NH₂-ATCTTAAATCGCAAGGT-3′ |
| | lized | |
| | probe 3 | |
| | Transi- | 5′-TGCTCAGTGTAGCCCAACCTTGCGATTTAAGAT- |
| | tion | 3′ |
| | probe 3 | |
| | Chromo- | 5′ TGGGCTACACTGAGCA-Biotin-3′ |
| | genic | |
| | probe 1 | |
| 8 | Immobi- | 5′-NH₂-ATCTTAAATCGCAAGGT-3′ |
| | lized | |
| | probe 3 | |
| | Trans- | 5′- |
| | ition | TGACTGCGAGTAGTAGCCAACCTTGCGATTTAAGAT- |
| | probe | 3′ |
| | 3-2 | |
| | Chromo- | 5′ TGGCTACTACTCGCAGTCA-Biotin-3′ |
| | genic | |
| | probe 2 | |
| 9 | Immobi- | 5′-NH₂-ATCTTAAATCGCAAGGT-3′ |
| | lized | |
| | probe 3 | |
| | Transi- | 5′-TCTCAGGTACCA ACCTTGCGATTTAAGAT-3′ |
| | tion | |
| | probe | |
| | 3-3 | |
| | Chromo- | 5′ TGGTACCTGAGA-Biotin-3′ |
| | genic | |
| | probe 3 | |
| 10 | Immobi- | 5′-NH₂-ATCCCGAAGGTGGTTAC-3′ |
| | lized | |
| | probe 4 | |
| | Transi- | 5′-GGTACCATCTCA GTAACCACCTTCGGGAT-3′ |
| | tion | |
| | probe 4 | |
| | Chromo- | 5′ TGAGATGGTACC-Biotin-3′ |
| | genic | |
| | probe 4 | |

2. Preparation of Detection Membranes

A nylon membrane enriched with carboxyl groups on the surface was talored into a required size, treated with 0.1 M HCl, and washed; the membrane was incubated in 15% EDC for 1 h and washed; then the membrane was incubated in 0.5 M NaHCO₃ solution containing 0.03 μM immobilized probe (probe combination 1 in Table 1) for 20 min; the membrane was then incubated in 0.2 M NaOH solution for 15 min, washed and dried. The thus prepared detection membranes were arranged on the targets to be sprayed for collecting spray droplets and the subsequent detection.

3. Preparation and Application of Spray Liquids Formulations to be sprayed (pesticide formulations, liquid fertilizers, other liquid formulations or water) were added into dosing tanks, and then transition probes (in water) were added. The final concentration of transition probes in the spray liquids was 0.60 μM. To each spray liquid, only one transition probe was added, and the transition probes were different from each other. Finally, based on the requirements of pesticide formulations, liquid fertilizers or spraying device, a surfactant and ionic buffer were added to prepare transition probe spray liquids (0.02-0.045 mol/L ion buffer and 0.06-0.15% surfactant). The major formulations were water-based formulation and oil-based formulation. In the preparation process of spray liquids in Example 1, the components of the spray liquids were 30 mM trisodium citrate, 3 mM SDS, and 0.06 μM transition probe. Probe combination 1 in Table 1 was selected, namely, transition probe 1 was used. After spraying, the detection membranes were retrieved and subjected to chromogenic reaction.

4. Establishment of Standard Curve

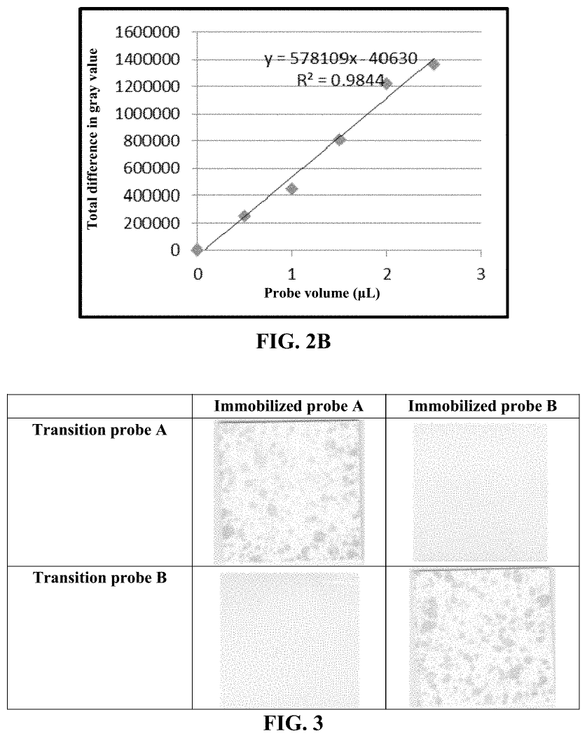

5 detection membranes containing immobilized probes were selected, 0.5 μL of spray liquid containing transition probe was applied to 1-5 spots on the 5 detection membranes respectively, wherein the volumes of spray liquid containing transition probe on the 5 detection membranes were 0.5 μL, 1.0 μL, 1.5μL, 2.0μL and 2.5μL, respectively. Another detection membrane was taken as background. An image file was obtained by photographing or scanning (FIG. 2A). Gray values of unit areas were obtained through an image processing software (for example, Photoshop, Image J), and a total gray value of a selected area was calculated. Finally, a standard curve of total gray value as the ordinate against the volume of spray as the abscissa was plotted, and a corresponding linear equation was calculated. The results are shown in FIG. 2B.

5. Chromogenic Treatment

Detection membranes sprayed with the spray liquids containing the transition probes were incubated in 50 mL of hybridization buffer (an aqueous solution containing 30 mmol/L trisodium citrate and 26 mmol/L SDS) at 37° C. for 40 min; after removing the hybridization buffer, another 50 mL of hybridization buffer was added to wash the detection membranes for 2 min; the detection membranes were then transferred to hybridization buffers containing chromogenic probes at 37° C. for reaction for 15 min, and were washed 3 times with 50 mL of washing buffer (an aqueous solution containing 7.5 mmol/L trisodium citrate and 6 mmol/L SDS) and once with 50 mL of hybridization buffer. 15 μL of streptavidin-labeled horseradish peroxidase was added into a hybridization buffer to prepare an enzyme solution; the detection membranes were incubated in the enzyme solution for enzyme-linked reaction at 37° C. for 20 min; the detection membranes were washed with 50 mL of hybridization buffer, and transferred into a TMB single-component solution for chromogenic reaction, wherein the TMB single-component solution was catalyzed by the horseradish peroxidase bound to the detection membrane, and then color developed on the detection membrane; and after 3 min, the membranes were washed with water to terminate the reaction, and dried. Information such as the distribution and size of droplets were directly observed through the chromogenic reaction. Finally, an image file was obtained by photographing or scanning. Gray values of unit areas were obtained through an image processing software (for example, Photoshop, Image J and the like), and a total gray value of a selected area was calculated.

### Example 2. Spray Boom Track System-Simulated Field Spraying—Simultaneous Detection by Two Probes

Two detection membranes (hereinafter referred to as detection membrane A and detection membrane B) respectively containing immobilized probe 1 and immobilized probe 2 (combination 1 and combination 4) were prepared according to the method of Example 1. Spray liquids containing transition probes (spray liquid A and spray liquid B) corresponding to the above immobilized probes were prepared according to the method of Example 1 for preparing the spray liquids. Culture dishes were placed on a support just below the middle of the pathway of the spray boom track system, with each culture dish containing 2 detection membranes (one each for A and B). Sprays were applied to the detection membranes under a pressure of 3 bar by the spray crane (speed: 5 km/h, height: 0.5 m) equipped with Lechler ST110-03 standard fan-shaped nozzle, with spray liquids A and B being applied once separately. After spraying, the experimental materials were retrieved, and the detection membranes were subjected to chromogenic reaction with the corresponding chromogenic probes according to the chromogenic treatment described above. The droplet coverage areas on the detection membranes were read by an instrument, and the droplet volume and coverage rate were calculated. Digital images were obtained by approaches such as photographing or scanning. Gray values of unit areas were obtained by an image processing software (for example, Photoshop, Image J and the like), and a total gray value of a selected area was calculated. The deposition volume was calculated from a standard curve. The results are shown in FIG. 3 and Table 2. From the results in FIG. 3, there was no interference between the 2 probe combinations, and the droplets of multiple sprays were simultaneously detected. Furthermore, the results in Table 2 show that the deposition volume of the droplets was accurately quantified.

TABLE 2
Experiment for simultaneously detecting
droplet properties by two probes

| Probe | A | B |
|---|---|---|
| Deposition volume (μL/cm²) | 2.40 | 2.06 |
| Theoretical deposition volume (μL/cm²) | 2.86 | 2.86 |
| Ratio (calculated/theoretical) | 0.84 | 0.72 |

### Example 3. Spray Boom Track System-Simulated Field Spraying—Simultaneous Detection by Three Probes

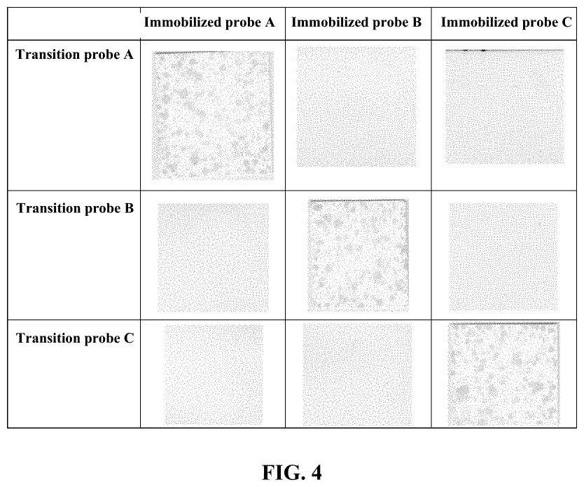

Three detection membranes (hereinafter referred to as detection membrane A, B and C) respectively containing immobilized probes 1, 2 and 3 (combinations 1, 4 and 7) were prepared according to the method as described above. Spray liquids containing transition probes (spray liquids A, B and C) corresponding to the above immobilized probes were prepared according to the aforementioned method for preparing the spray liquids. Culture dishes were placed on a support below the pathway of the spray boom track system and just in the middle of the pathway of the spray boom track system, with each culture dish containing 3 detection membranes (one each for A, B and C). Sprays were applied to the detection membranes under a pressure of 3 bar by the spray boom track system (speed: 5 km/h, height: 0.5 m) equipped with Lechler ST110-03 standard fan-shaped nozzle, with spray liquids A, B and C being applied once separately. After spraying, the experimental materials were retrieved, and the detection membranes were subjected to chromogenic reaction with the corresponding chromogenic probes according to the chromogenic treatment described above. The droplet coverage areas on the detection membranes were read by an instrument, and the droplet volume and coverage rate were calculated. Digital images were obtained by approaches such as photographing or scanning. Gray values of unit areas were obtained by an image processing software (for example, Photoshop, Image J and the like), and a total gray value of a selected area was calculated. The deposition volume was calculated from a standard curve. The results are shown in FIG. 4 and Table 3. From the results in FIG. 4, there was no interference between the 3 probe combinations, and the droplets of multiple sprays were simultaneously detected. Furthermore, the results in Table 2 show that the deposition volume of the droplets was accurately quantified.

TABLE 3
Experiment for simultaneously detecting
droplet properties by three probes

| Probe | A | B | C |
|---|---|---|---|
| Deposition volume (μL/cm²) | 2.23 | 1.97 | 1.94 |
| Theoretical deposition volume (μL/cm²) | 2.86 | 2.86 | 2.86 |
| Ratio (calculated/theoretical) | 0.78 | 0.69 | 0.68 |

### Example 4. Sprayboom Track System-Simulated Field Spraying—Simultaneous Detection by Four Probes

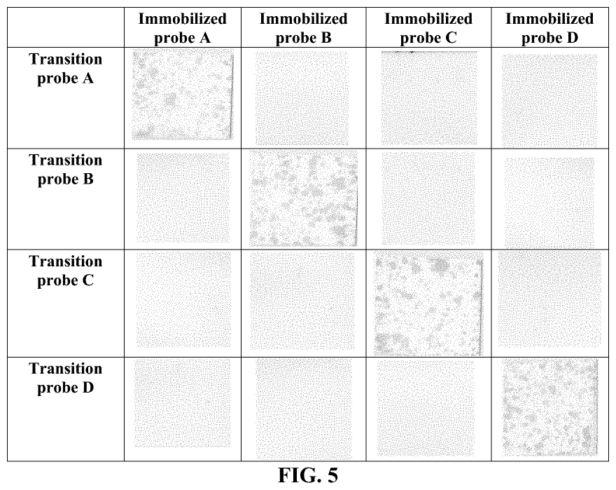

Four detection membranes (hereinafter referred to as detection membranes A, B, C and D) respectively containing immobilized probes 1, 2, 3 and 4 (combinations 1, 4, 7 and 10) were prepared according to the method as described above. Spray liquids containing transition probes (spray liquids A, B, C and D) corresponding to the above immobilized probes were prepared according to the aforementioned method for preparing the spray liquids. Culture dishes were placed on the iron support below the pathway of the spray boom track system and just in the middle of the pathway of the spray boom track system, with each culture dish containing 4 detection membranes (one each for A, B, C and D). Sprays were applied to the detection membranes under a pressure of 3 bar by the spray boom track system (speed: 5 km/h, height: 0.5 m) equipped with Lechler ST110-03 standard fan-shaped nozzle, with spray liquids A, B, C and D being applied once separately. After spraying, the experimental materials were retrieved, and the detection membranes were subjected to chromogenic reaction with the corresponding chromogenic probes according to the chromogenic treatment as described above. The droplet coverage areas on the detection membranes were read by an instrument, and the droplet number and coverage rate were calculated. Digital images were obtained by approaches such as photographing or scanning. Gray values of unit areas were obtained by an image processing software (for example, Photoshop, Image J and the like), and a total gray value of a selected area was calculated. The deposition volume was calculated from a standard curve. The results are shown in FIG. 5 and Table 4. From the results in FIG. 5, there was no interference between the 4 probe combinations, and the droplets of multiple sprays were simultaneously detected. Furthermore, the results in Table 3 show that the deposition volume of the droplets was accurately quantified.

TABLE 4
Experiment for simultaneously detecting
droplet properties by four probes

| Probe | A | B | C | D |
|---|---|---|---|---|
| Deposition volume | 2.06 | 1.97 | 2.23 | 2.40 |
| (μL/cm²) | | | | |
| Theoretical deposition | 2.86 | 2.86 | 2.86 | 2.86 |
| volume (μL/cm²) | | | | |
| Ratio | 72% | 69% | 78% | 84% |
| (calculated/theoretical) | | | | |

1. A method for simultaneously detecting the droplet drift or deposition volumes of multiple sprays, comprising:
(1) adding different transition probes into multiple spray liquids respectively as tracers to give spray liquids containing transition probes, wherein only one transition probe is added to each spray liquid;
(2) applying the spray liquids containing transition probes, then the transition probes in the spray liquids specifically bind to the corresponding immobilized probes on the detection membranes, wherein the detection membranes are substrates carrying the immobilized probes;
(3) adding biotinylated chromogenic probes, then they binding to the corresponding transition probes through hybridization, after the chromogenic treatment, determining the volume of droplets according to the color depth, and determining the droplet drift or deposition volume according to the location and size of colored spots;
wherein the transition probes are not biotinylated, but have nucleotide sequences capable of complementarily pairing with the corresponding immobilized probes and the corresponding chromogenic probes; the immobilized probes do not specifically bind to the chromogenic probes, and different transition probes do not specifically bind to each other.

2. The method according to claim 1, wherein the transition probes and the immobilized probes are single-stranded deoxyribonucleic acids with characteristic sequences; the length of the transition probes is 24-50 nt, and the length of the immobilized probes is 12-25 nt; one end of the immobilized probes is amino-modified and covalently binds to an exposed carboxyl of the substrate.

3. The method according to claim 1, wherein the complementary pairing region of the chromogenic probe and the transition probe is of 15-40 nt; if the immobilized probe is 5′-labeled, the chromogenic probe is 3′-biotinylated, and if the immobilized probe is 3′-labeled, the chromogenic probe is 5′-biotinylated.

4. The method according to claim 1, wherein the length of the immobilized probes is 18-20 nt, and the complementary pairing region of the transition probe and the immobilized probe is of 15-25 nt.

5. The method according to any of claims 1-4, wherein the detection membrane in step (2) is prepared according to the following method: acquiring a substrate of a required area, treating the substrate with 0.1-0.3 M HCl, and washing; incubating the substrate in 10-20% EDC solution and washing; incubating the substrate in 0.3-1.0 M NaHCO₃ solution containing 0.025-0.2 μM immobilized probe; and incubating the treated substrate in NaOH solution, washing and drying.

6. The method according to claim 5, wherein the detection membrane is prepared according to the following method: acquiring a substrate of a required area, treating the substrate with 0.1 M HCl, and washing; incubating the substrate in 15% EDC for 0.5-1 h and washing; incubating the substrate in 0.5 M NaHCO₃ solution containing 0.03 μM immobilized probe for 10-20 min; and incubating the treated substrate in 0.05-0.5 M NaOH solution for 5-15 min, washing and drying.

7. The method according to claim 5, wherein the substrate is a nitrocellulose membrane, a nylon membrane, a carboxylated organic glass film or a carboxylated polypropylene plastic film.

8. The method according to any of claims 1-4, wherein the final concentration of the transition probe in the spray liquid containing the transition probe in step (2) is 0.025-0.1 μM.

9. A kit for simultaneously detecting the droplet drift or deposition volumes of multiple sprays, comprising detection membranes, transition probes and chromogenic probes, wherein the numbers of the detection membranes, the transition probes and the chromogenic probes are all >2 and are different; the 3′ or 5′ end of the chromogenic probes is biotinylated, and the chromogenic probes can specifically bind to the transition probes but cannot specifically bind to the immobilized probes;
preferably, the detection membrane is a substrate fixed with the immobilized probe; the length of the immobilized probe is 12-25 nt; one end of the immobilized probe is amino-modified and covalently binds to an exposed carboxyl group of the substrate; the substrate is a material with exposed carboxyl groups;
preferably, the length of the transition probe is 24-50 nt.

10. The kit according to claim 9, further comprising a TMB (3,3′,5,5′-tetramethylbenzidine) single-component solution, and streptavidin-labeled horseradish peroxidase.
